(19) United States
(12) Patent Application Publication
(10) Pub. No.: US 2009/0012522 A1
(43) Pub. Date: Jan. 8, 2009

(54) Fixing Element

(76) Inventor: Gunter Lob, Munich (DE)

Correspondence Address: WARE FRESSOLA VAN DER SLUYS & ADOLPHSON, LLP, BRADFORD GREEN, BUILDING 5, 755 MAIN STREET, P O BOX 224, MONROE, CT, 06468, US

(21) Appl. No.: 11/884,336

(22) PCT Filed: Feb. 23, 2006
(86) PCT No.: PCT/EP06/60217

§ 371 Date: Sep. 12, 2008

(30) Foreign Application Priority Data
Feb. 24, 2005 (DE) 10 2005 009 492.9

(51) Int. Cl. A61B 17/68

(52) U.S. Cl. 606/60

(57) ABSTRACT

A fixing element, in particular a peg for fixing bone fragments or the like, is divided in the longitudinal direction into at least two sub-elements which jointly enclose an expansion element. The sub-elements and the expansion element are of such a configuration and are arranged relative to each other in such a way that the expansion element is disposed between the sub-elements and a displacement of the expansion element in the longitudinal direction of the fixing element causes a displacement of the sub-elements in the lateral direction. In this way, the sub-elements are urged away from each other in the lateral direction and the fixing element is thereby transferred from a closed condition into an expanded condition. The fixing element has a distal longitudinal end and a proximal longitudinal end and is to be inserted with its distal longitudinal end leading. The fixing element has at least one traction element actuated from the proximal end of the fixing element and at least temporarily connected to the expansion element in such a way that actuation of the at least one traction element by pulling on the at least one traction element causes the longitudinal displacement of the expansion element, which causes the expansion of the fixing element.

### BACKGROUND OF THE INVENTION

1. Field of the Invention

The invention concerns a fixing element, in particular a peg, for fixing for example bone fragments, that is to say a fixing element for surgical use.

2. Description of the Background Art

The fixing element is divided in the longitudinal direction into at least two sub-elements which by virtue of the division are movable relative to each other transversely with respect to the longitudinal direction, that is to say in a lateral direction. The sub-elements enclose an expansion element. The expansion element and the sub-elements are of such a configuration and are arranged relative to each other in such a way that the expansion element is disposed between the sub-elements and displacement of the expansion element in the longitudinal direction of the fixing element causes a displacement of the sub-elements in the lateral direction so that, by virtue of the displacement of the expansion element, the sub-elements are driven away from each other in the lateral direction and the fixing element is thereby changed from a closed condition into a spread condition.

A fixing element of that kind is known for example from WO96/16607.

Fixing elements of that kind are used for example in the manner of a peg for fixing bone elements. For that purpose the fixing element is inserted into a bore and then expanded by means of the expansion element. By virtue of the expansion effect the fixing element is fixed to the two bone fragments and in that way also fixes the two bone elements relative to each other.

### SUMMARY OF THE INVENTION

In consideration of the state of the art there is a need to improve the handling of the fixing element.

In accordance with the invention that need is met with a fixing element having a traction element which is to be actuated from the proximal end of the actuating element and which is at least temporarily connected to the expansion element in such a way that actuation of the traction element by pulling on the traction element causes the longitudinal displacement of the expansion element, which causes the expansion of the fixing element.

An expansion element which is to be actuated in retrograde fashion by way of a traction element involves some advantages. Thus, for example, expansion of the fixing element can be effected without force components acting in the meantime on the body of the patient in the longitudinal direction of the fixing element. That is possible as a pressure force can be exerted on the proximal end of the fixing element, that pressure force corresponding to the tensile force exerted on the traction element so that the two forces compensate each other.

In addition, the traction element can be in the form of a wire, cord or also in the form of a surgical thread. In the last-mentioned case, the instruments which already exist and which for example make it easier to suture wounds can also be used for tensioning and then knotting the traction element.

Accordingly, the traction element is preferably in the form of a thread, for example surgical thread, wire or cord.

The expansion element can in principle be of many different configurations. Thus it is possible for the fixing element to be divided along its longitudinal direction into four sub-elements which are distributed uniformly around the periphery of the fixing element. Each of those four sub-elements can be for example of a cross-sectional shape which corresponds to that of a quarter of a circle which at its center has an opening which, when viewed in conjunction with the openings at the other three sub-elements, affords a rectangular quadrangle. That rectangular quadrangle can then be cleverly oriented in such a way that its diagonals extend in the direction of the angle bisectors of the cross-section, in the shape of a quarter of a circle, of a respective sub-element. The expansion element can then be of a pyramid shape, wherein a respective one of the edges converging to the tip of the pyramid extends in a groove afforded by the opening in the cross-section of a respective sub-element.

The configuration of the sub-elements and the expansion element is such that the expansion element, both in the closed condition of the fixing element and also in the expanded condition thereof, does not project beyond the proximal end of the fixing element, which is defined by the sub-elements, or beyond the distal end of the fixing element, which is defined by the sub-elements, but rather is completely enclosed by both sub-elements in any condition of the fixing element.

Irrespective of that lengthwise dimension according to the invention, it is an independent concept of the invention for the groove for the traction element to be of such a dimension that the traction element is freely movable in the longitudinal direction of the traction element in any condition of the fixing element between its sub-elements and the expansion element.

Preferably the fixing element has two sub-elements. In that case the expansion element is preferably of a wedge-shaped configuration and narrows in the proximal direction of the fixing element. The two sub-elements then preferably each have a respective inwardly directed sliding surface which corresponds to a wedge surface of the expansion element and which in the closed condition of the fixing element extends parallel to the respective wedge surface. That provides that, upon actuation of the traction element the fixing element is uniformly expanded at the proximal end and also at the distal end.

It is precisely the fact that the expansion element is in the form of a relatively pointed and thus shallow wedge that makes it possible for a traction element in the form of a thread, cord or wire to be laid around the distal end of the expansion element and allows the traction element to be guided from there along the two wedge surfaces so that the two free ends of the traction element protrude beyond the proximal end of the fixing element and can be actuated from there.

In order to fix the traction element which is laid around the wedge-shaped expansion element in regard to the position of the traction element and in order to permit the respective wedge surface of the expansion element to act directly on the respective sliding surface of the sub-element without the traction element impeding direct contact and without the traction element being clamped in its longitudinal direction, preferably at least one groove extending in the longitudinal direction of the fixing element is provided on the outside of the expansion element, to receive the traction element. A respective groove in a wedge surface is preferably provided around the distal end of the expansion element and then further continues in the respective other wedge surface. That affords a continuous, peripherally extending groove. Preferably there are two such grooves so that the fixing element can also have two traction elements in the form for example of a thread.

Instead of providing grooves in the expansion element the grooves can also be provided on the insides of the sub-elements of the fixing element in those surfaces along which a respective expansion element slides upon expansion of the fixing element.

In any case—that is to say irrespective of the form of the expansion element or on the expansion element or the sub-elements of the fixing element, which are to be expanded—corresponding grooves for the traction element or elements are preferably of such dimensions that a respective traction element, for example a thread, can be freely moved both in the unexpanded condition of the fixing element and also in the expanded condition thereof. More specifically that means that the two thread ends which project proximally out of the expanded fixing element after the implantation thereof can be ideally knotted without the knotting operation being impeded by the thread being fixed for example by being clamped. Complete removal of the traction element is also possible in that way after expansion (spreading) of the fixing element. The latter can be of advantage in particular when the wedge angle between the wedge surfaces of the expansion element and the sliding surfaces of the sub-elements of the fixing element are of such dimensions that the expansion element is self-locking by virtue of the frictional forces operative between wedge surfaces and sliding surfaces, so that after expansion the expansion element cannot slide back independently without further axial forces acting thereon into its starting position again in the unexpanded closed condition of the fixing element.

In the closed condition, the fixing element is preferably in the form of a cylinder with projections extending in the peripheral direction and recesses correspondingly extending between the projections in the peripheral direction. A cylindrical external shape for the fixing element has the advantage that appropriately matching round bores are easy to produce, in bone fragments which are to be fixed to each other. The projections extending in the peripheral direction counteract slippage of the fixing element in the longitudinal direction thereof while the fixing element is being expanded or after it has been expanded.

Finally it is desirable if the sub-elements of the fixing element cannot be displaced relative to each other with respect to the longitudinal direction of the fixing element during expansion of the fixing element or also at the actual operation of inserting the fixing element into a corresponding bore. In order to prevent such relative longitudinal displacement, it is provided that a separation surface between the sub-elements, along which the sub-elements bear against each other in the closed condition of the fixing element, has a step in the peripheral direction. That step in the separation surface provides that there is at least one separation surface portion which extends transversely with respect to the longitudinal direction of the fixing element and which counteracts displacement of the sub-elements in the longitudinal direction relative to each other.

The sliding surface of a respective sub-element is preferably the bottom of a recess let into the respective separation surface of the sub-element. That recess is preferably of a rectangular cross-section. In a corresponding fashion, the cross-sections of the expansion element, which extend transversely with respect to the longitudinal direction of the fixing element, are also preferably rectangular. The two wedge surfaces of the expansion element then adjoin along their longitudinal edges longitudinal side surfaces of the expansion element, which extend in mutually parallel relationship. At its distal end, the expansion element is preferably rounded in a semicylindrical configuration, more specifically in such a way that the two wedge surfaces of the expansion element go into the peripheral surface of the semicylindrical shape, at their distal end. If, in accordance with a preferred variant, the traction element is passed around the distal end of the expansion element, the semicylindrical shape affords the advantage that the bending radius of the traction element in that region can be kept as large as possible so that no stress peaks occur, which promote tearing of the traction element. The semicylindrical shape of the distal end of the expansion element further supports the above-mentioned advantageous free mobility of the traction element in the respective longitudinal direction thereof.

Finally, the expansion element is advantageously of such a dimension in the longitudinal direction that, even in the condition in which the fixing element is expanded to the maximum degree, it does not project proximally out of the fixing element. The expansion element equally does not project from its distal end when the fixing element is closed. Rather, the expansion element is preferably disposed completely within the fixing element in any condition of the fixing element.

Further advantageous design features of the expansion element are to be found in the claims and the description hereinafter.

In relation to the intended use of the fixing element, namely joining bone fragments which are to be fixed together by means of the fixing element in such a way that they then grow together again in a natural healing process, it is particularly advantageous if the fixing element is resorbed by the body in the course of time. Therefore the component parts of the fixing element preferably comprise a bioresorbable plastic material.

### BRIEF DESCRIPTION OF THE DRAWINGS

The invention will now be described in greater detail by means of an embodiment by way of example illustrated in a number of Figures in which:

FIG. 1 is a perspective view of the two sub-elements and the expansion element of a fixing element of the kind according to the invention,

FIG. 2 is an exploded perspective view of the component parts of the fixing element of FIG. 1,

FIG. 3 is a perspective view of the fixing element of FIGS. 1 and 2 with the sub-elements assembled,

FIG. 4 is a perspective view of the fixing element according to the invention in the closed condition,

FIG. 5 is a longitudinal section through the fixing element in the condition shown in FIG. 4,

FIG. 6 is a perspective view of the fixing element in the expanded condition, and

FIG. 7 is a longitudinal section through the fixing element in the expanded condition of FIG. 6.

### DETAILED DESCRIPTION OF THE INVENTION

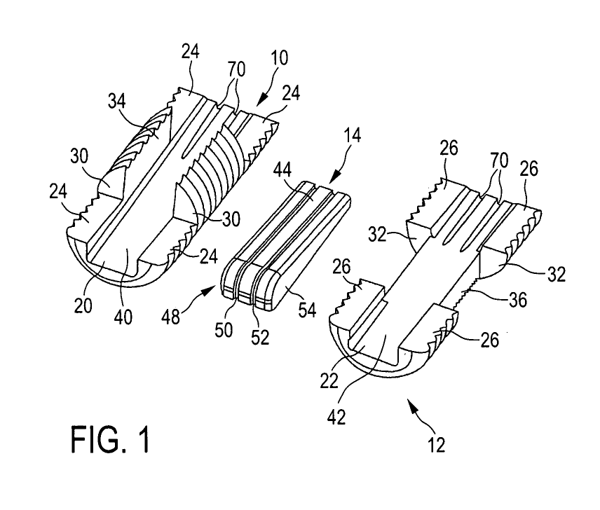

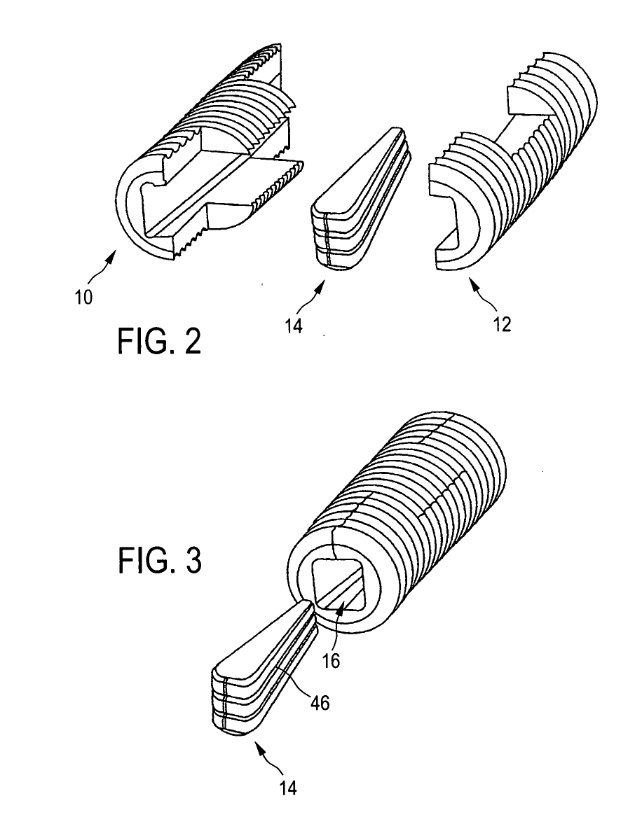

FIG. 1 shows the component parts of a fixing element according to the invention with the exception of a thread serving as a traction element. These component parts are a first sub-element 10, a second sub-element 12 and an expansion element 14. As can be seen from FIG. 3, the two sub-elements 10 and 12 can be assembled in such a way that the fixing element is of a cylindrical external shape. The two sub-elements 10 and 12 then enclose between them a wedge-shaped opening 16 into which the expansion element 14 precisely fits.

The wedge-shaped opening 16 is formed by recesses 20 and 22 in the respective sub-element 10 and 12. The recesses 20 and 22 are disposed on the inside of a respective sub-element 10 or 12 and are recesses with respect to a separation surface which extends substantially parallel to the longitudinal direction of the fixing element. The separation surface leads to separation sub-surfaces 24 and 26 respectively on the sub-elements 10 and 12 respectively which in the closed condition of the fixing element bear against each other. In a central longitudinal portion of the fixing element the separation surface has a step so that separation surface portions 30 and 32 extending transversely with respect to the longitudinal direction of the fixing element are provided on the sub-elements 10 and 12 respectively. Those separation surface portions 30 and 32 provide that the sub-elements 10 and 12 cannot be displaced relative to each other in both the closed and the expanded condition of the fixing element. Separation surface portions 34 and 36 in parallel relationship with the longitudinal axis also prevent the sub-elements 10 and 12 from being displaceable laterally relative to each other, more specifically even when the expansion element 14 is not inserted into the wedge-shaped opening 16 enclosed by the two sub-elements 10 and 12.

As can be clearly seen in particular from FIG. 1, the respective recesses in the sub-elements 10 and 12 each have a floor serving as a sliding surface 40 and 42 respectively.

When the expansion element 14 is inserted, those sliding surfaces 40 and 42 co-operate with corresponding wedge surfaces 44 and 46 of the expansion element 14.

At their distal end, the wedge surfaces 44 and 46 of the expansion element 14 form a transition into a semicylindrical shape 48 of the expansion element. Two grooves 50 and 52 each extend in the longitudinal direction from one wedge surface over the semicylindrical shape to the other wedge surface. The grooves are of such a size that they can completely accommodate a thread serving as a traction element so that a longitudinal movement of the thread between the sliding surfaces 40 and 42 and the respectively oppositely disposed wedge surfaces 44 and 46 is not impeded.

The wedge surfaces 44 and 46 and the semicylindrical shape 48 form a transition laterally into longitudinal side surfaces 54 of the expansion element 14. The edges between the wedge surfaces or the semicylindrical shape and the longitudinal side surfaces 54 of the expansion element 14 are rounded.

The grooves 50 and 52 serve to accommodate a respective surgical thread as the traction element.

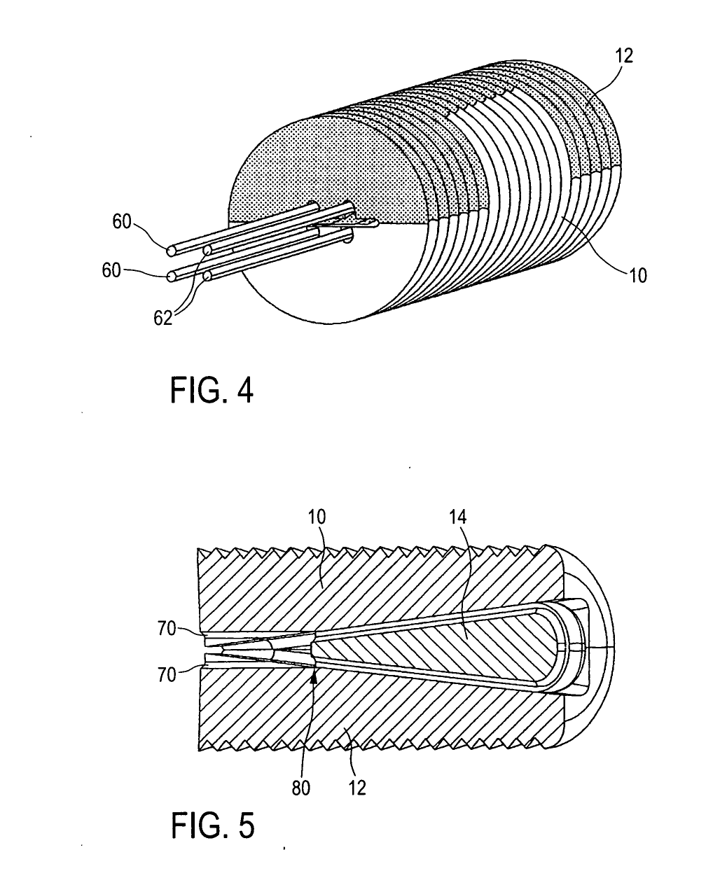

That will be readily understood when viewing FIGS. 4 through 7.

FIG. 4 shows the expansion element in the closed condition with the expansion element inserted but not visible. The expansion element is enclosed over its entire length between the two sub-elements 10 and 12, as FIG. 5 also shows. In contrast, two free ends 60 and 62 respectively of two surgical threads serving as traction elements are respectively visible.

It will be seen from FIG. 7 which shows a view in longitudinal section through the fixing element of FIG. 4 in the closed condition how the surgical thread as the traction element passes around the expansion element 14 and is let into the corresponding groove of the expansion element in such a way that it can move freely in the longitudinal direction between the expansion element 14 and the sub-elements 10 and 12.

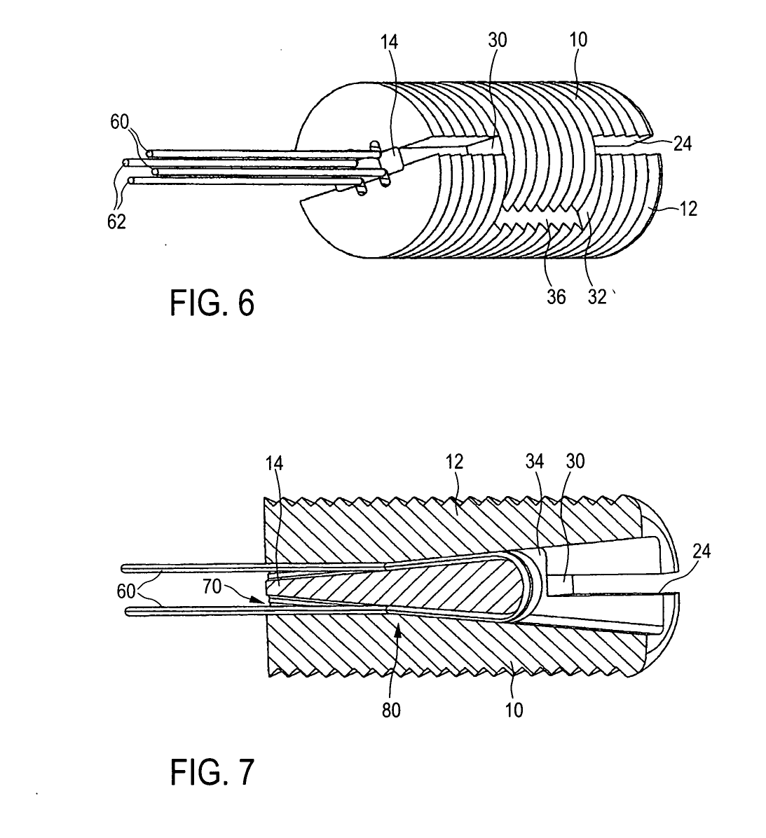

By pulling on the surgical threads serving as the traction element, the expansion element 14 is drawn deeper into the opening 16 and in that case urges the two sub-elements 10 and 12 laterally away from each other so that the expansion element is expanded as shown in FIG. 6. FIG. 7 shows the associated longitudinal section of the fixing element in the fully expanded condition. In that condition the proximal end of the expansion element 14 is in flush terminating relationship with proximal end faces of the sub-elements 10 and 14 without protruding beyond them.

As can be seen in particular from FIGS. 5 and 7 provided at the proximal end of the sliding surfaces in the sub-elements 10 and 12 are short groove portions 70 which correspond in their position to the grooves in the expansion element 16 and which provide that the surgical threads can already extend straight from the positions indicated at 80 in FIGS. 5 and 7.

As can be seen in particular from FIGS. 3 and 4 the fixing element is ribbed on its peripheral surface. That ribbing derives from a plurality of projections which extend therearound in the peripheral direction and which serve for anchoring the fixing element in the longitudinal direction.

What is claimed is:

1-18. (canceled)

19. A fixing element for fixing bone fragments or the like and having a distal longitudinal end and a proximal longitudinal end and to be inserted with its distal longitudinal end leading, the fixing element comprising:
(a) at least two sub-elements dividing the fixing element in a longitudinal direction;
(b) an expansion element enclosed by the at least two sub-elements, wherein the sub-elements and the expansion element are of such a configuration and are arranged relative to each other in such a way that the expansion element is disposed between the sub-elements and a displacement of the expansion element in the longitudinal direction of the fixing element causes a displacement of the sub-elements in a lateral direction so that the sub-elements are urged away from each other in the lateral direction and the fixing element is caused to expand and thereby transferred from a closed condition into an expanded condition; and
(c) at least one traction element adapted to be actuated from the proximal end of the fixing element and at least temporarily connected to the expansion element in such a way that actuation of the at least one traction element by pulling on the at least one traction element causes the longitudinal displacement of the expansion element, which causes the expansion of the fixing element, wherein both in the closed condition of the fixing element and also in the expanded condition thereof the expansion element does not project beyond the proximal end of the fixing element, which is defined by the sub-elements, or beyond the distal end of the fixing element, which is defined by the sub-elements.

20. A fixing element as set forth in claim 19, wherein each traction element is a thread, wire, cord or the like.

21. A fixing element as set forth in claim 20, wherein each traction element is laid in the manner of a loop around the distal end of the expansion element so that both free ends of each traction element are at the proximal end of the fixing element.

22. A fixing element as set forth in claim 21, wherein, on its outside, the expansion element has at least one groove extending in the longitudinal direction of the fixing element, within which is received the at least one traction element.

23. A fixing element as set forth in claim 22, wherein the at least one groove extends along both wedge surfaces and is passed around the distal end of the expansion element.

24. A fixing element as set forth in claim 22, wherein the expansion element has two grooves extending in mutually parallel relationship.

25. A fixing element as set forth in claim 22, wherein each groove is dimensionally sized to completely accommodate a respective traction element to move freely in the groove in the longitudinal direction in any condition of the fixing element.

26. A fixing element as set forth in claim 19, wherein, in its closed condition, the fixing element is in the form of a cylinder with projections and recesses respectively extending in a peripheral direction.

27. A fixing element as set forth in claim 19, wherein the sub-elements are separated from each other along a separation surface and said separation surface steps in the longitudinal direction of the fixing element so that there is at least one separation surface portion which extends transversely with respect to the longitudinal direction of the fixing element and which also counteracts displacement of the sub-elements relative to each other with respect to the longitudinal direction of the fixing element upon expansion of the fixing element.

28. A fixing element as set forth in claim 19, wherein the expansion element is of a wedge-shaped configuration and narrows in the proximal direction of the fixing element.

29. A fixing element as set forth in claim 28, wherein the sub-elements each have a respective inwardly directed sliding surface which respectively corresponds to a wedge surface of the expansion element and which in the closed condition of the fixing element extends in parallel relationship with respective wedge surface so that upon actuation of the traction element the fixing element is equally expanded at the proximal end and at the distal end.

30. A fixing element as set forth in claim 29, wherein the sliding surface of a respective sub-element is a bottom wall of a recess.

31. A fixing element as set forth in claim 28, wherein the cross-sections of the expansion element, which extend transversely with respect to the longitudinal direction of the fixing element, are each of a generally rectangular shape.

32. A fixing element as set forth in claim 31, wherein the expansion element has two wedge surfaces which, along their longitudinal edges, adjoin longitudinal side surfaces of the expansion element, which extend in mutually parallel relationship.

33. A fixing element as set forth in claim 28, wherein, at its distal end, the expansion element is rounded in a semicylindrical configuration, wherein a peripheral surface of the semicylindrical shape goes into respective wedge surfaces of the expansion element.

34. A fixing element as set forth in claim 33, wherein end faces of the semicylindrical shape respectively form a distal portion of flat longitudinal side surfaces of the expansion element.

35. A fixing element as set forth in claim 33, wherein the semicylindrical shape defines at least one groove, each being an extension of a corresponding groove in the respective wedge surfaces of the expansion element.

36. A fixing element as set forth in claim 19, wherein the two sub-elements and the expansion element are comprised of bioresorbable plastic material.
